(19) United States
(12) Patent Application Publication
(10) Pub. No.: US 2003/0176785 A1
(43) Pub. Date: Sep. 18, 2003

(54) Method and apparatus for emergency patient tracking

(75) Inventors: Robert F. Buckman, Radnor, PA (US); Jay A. Lenker, Laguna Beach, CA (US)

Correspondence Address: Crockett & Crockett, Suite 400, 24012 Calle De La Plata, Laguna Hills, CA, 92653, US

(73) Assignee: Damage Control Surgical Technologies, Inc.

(21) Appl. No.: 10/358,896

(22) Filed: Feb. 4, 2003

Related U.S. Application Data
(60) Provisional application No. 60/354,430, filed on Feb. 4, 2002.

(51) Int. Cl. A61B 5/117

(52) U.S. Cl. 600/431

(57) ABSTRACT

A system is disclosed for tracking patients. Such systems are especially useful in the emergency or military setting or in the case of a national emergency. In such cases, the patient may be transported far from home or from the site of the injury in order to receive treatment. The system has application, as well however, for patient tracking within normal hospital systems. The system utilizes a passive, unique coding system for each patient. A coded device is associated with each patient in such a way that the coded device cannot be removed or disassociated from the patient without a concerted effort. A reader and encoder is used at the time of application of the coded device, to the patient, to enter pertinent information about the patient into a national database. Such information may be used or augmented as required during the medical care of the patient and return of the patient to normal life. Information gained from the device is also used to assist in prioritizing the patient regarding the order of triage relative to other patients. The reader and encoder communicate with a centralized database to enter information regarding the patient as well as the location of the patient.

This application claims priority benefit under 35 USC § 119(e) from U.S. Provisional Application No. 60/354,430 filed Feb. 4, 2002, entitled “METHOD AND APPARATUS FOR EMERGENCY PATIENT TRACKING” which is herein incorporated by reference.

### FIELD OF THE INVENTION

The field of this invention is emergency and trauma medicine.

### BACKGROUND OF THE INVENTION

During a time of national emergency or a state of war, large populations may be subject to injury. Since medical facilities in the affected area may be damaged or under-resourced, patients may need to be moved substantial distances in order to accommodate the medical needs of these large populations.

Current medical transponders are designed to track implants in humans or to track ruminating animals. Typical examples of the prior art in medial transponders include U.S. Pat. No. 4,160,971 to Jones et al., U.S. Pat. No. 5,300,120 to Knapp et al., U.S. Pat. No. 5,652,570 to Lepkofker, U.S. Pat. No. 5,674,288 to Knapp et al., U.S. Pat. No. 5,716,407 to Knapp et al., 5,725,578 to Knapp et al., U.S. Pat. No. 5,732,401 to Conway, 5,855,609 to Knapp, U.S. Pat. No. 5,977,431 to Knapp et al. and U.S. Pat. No. 6,012,415 to Linseth. These patents, all of which are included herein by reference, disclose medical transponders used in a variety of ways, either attached to implants, worn on the wrist, swallowed, or implanted separately from implants. These devices all serve the purpose of tracking patients (animals as in the case of U.S. Pat. No. 6,012,415) or the medical devices with which they are implanted. While transponders are well known in the art, the utility of these transponders for patient tracking in an emergency-situation has not been addressed.

New devices, systems and methods are needed to track patients, who are being treated at centers that may be remote from their home. Such devices are particularly important in an emergency setting or trauma setting, such as could occur during a national emergency or during military action.

### SUMMARY OF THE INVENTION

The system and method described below relate to a medical transponders and systems for tracking patients. The system uses a passive device that allows for patient identification and tracking when used in conjunction with a smart reader.

During a state of national emergency, hospitals may be elevated to a state of war. A network of hospitals may be enabled to care for the victims because of the immense number of victims, or the hospitals in the vicinity of the disaster may be dysfunctional or nonexistent. Patients may be transported far from their homes to obtain medical treatment within the hospital network. For this reason, patient identification is important to ensure maximum quality of care and to ensure that the patient is routed through the system properly and properly identified throughout the system (or their remains are returned to their families).

The invention involves use of a passive, unique identification that is associated with the patient. Such identification will contain a unique code, including bar codes, Arabic numerals and the like. The identifying marks are placed on the patient in an area that is not damaged and in an area that is easily reached and obvious to the medical personnel. The identifying mark would, by protocol, be preferentially placed on the head or neck of the patient. Should that area not be available, the mark would be placed at a location of secondary priority, such as the torso, shoulder, arm, or leg. Tertiary locations can also be identified and prioritized by protocol. The code is imprinted in a wrist band, leg band or other medical jewelry that is attached securely to the patient. By using passive identification, the system will not fail should batteries or capacitors become depleted of power. Therefore, the patient is always associated with a unique code.

Additional apparatus suitable for identifying the patient includes RF ID transponders that are attached to the patients wrist, neck, leg or to their ear via an earring. The transponder could also be placed subcutaneously using an injector. Again, according to protocol, the transponder would be placed at locations of descending priority, depending on the medical state of the patient. Typical locations for a transponder are in the abdominal, shoulder or thoracic area.

The information encoded by the system would minimally include a unique identifying number. This number is associated with that patient only and is the key to a larger database of information about the patient. Additional information could also be added to the transponder, tattoo or jewelry.

The person applying the coded transponder, mark or jewelry, hereinafter called the coded device, would receive the number from a centralized database that assigns such numbers so that duplicates would be avoided.

The information flow between the medical provider and the centralized database is facilitated by satellite uplink, cellular telephone system or other wireless communication system.

At the time the coded device is applied to the patient, certain data will be collected by the medical provider using a data encoding system. This data encoding system will include a timepiece to automatically record time and a GPS system to record exact location of pickup as well as a pre-assigned code for the medical team performing the initial triage and coded device application. A data input system will be provided to allow for input of vital medical data including patient temperature, ECG, and blood data. Additionally, a dictated statement regarding the patient's condition, circumstances of the patient, triage status and known and/or suspected injuries. This information will be sent to the centralized database and be associated with the patient.

In accordance with another aspect of the invention, a method is described to provide patient triage and management in a state of emergency. This method includes the steps of applying a coded device, with a unique code, to a patient, associating certain patient, geographic and time data with the unique code, and transmitting this information to a centralized database for future use in treating the patient.

In yet another embodiment of the invention, the patient status is ascertained using an ultrasonic instrument to determine and diagnose such things as, but not limited to, the status of such things as heart rate, blood pressure, ejection fraction, respiratory rate, quality of the breathing, respiratory volumes, and the presence or absence of a pneumothorax or tension pneumothorax. The device also provides a painful stimulus, preferably non-penetrating or non-invasive, to the patient, to determine cognitive status and/or the ability of the patient to localize the painful stimulus. Using this information, the device of this embodiment of the invention, evaluates the patient status and provides a numerical score regarding triage status that can be used to prioritize treatment of the patient relative to other patients who have sustained injuries. The triage status is based on-cognitive status, cardiovascular status, pulmonary status, and/or a combination thereof. The patient triage score is preferably based on consideration to those who have the highest probability of survival post-treatment, in a mass-casualty situation. This triage status and priority, as well as the data supporting the status and priority, are optionally transmitted to the centralized database. Phase-shifted calculations are used to look at probable mortality of the patients. Using this system, the mid-level casualties are the first to be moved, rather than the most-severely injured, because the most severely injured people will most likely die.

The present invention distinguishes over the cited prior art because it requires a uniquely coded device to be attached to the patient and it uses a specialized data encoding system to provide medical data to a centralized database, associated with the embedded code in the coded device on the patient. The invention is most useful during civil emergencies or military situations, when large numbers of patients require treatment, often at remote sites, and patient identification may be lost.

### BRIEF DESCRIPTION OF THE DRAWINGS

FIG. 1A illustrates a diagram of the coded device using a bracelet, according to aspects of an embodiment of the invention.

FIG. 1B illustrates a diagram of the coded device using an implanted transponder, according to aspects of an embodiment of the invention.

FIG. 1C illustrates a diagram of the coded device using a tattoo placed on the patient, according to aspects of an embodiment of the invention.

FIG. 2 illustrates a block diagram of the data encoding system, according to aspects of an embodiment of the invention.

FIG. 3 illustrates a block diagram of the centralized database system, according to aspects of an embodiment of the invention.

### DETAILED DESCRIPTION OF THE INVENTION

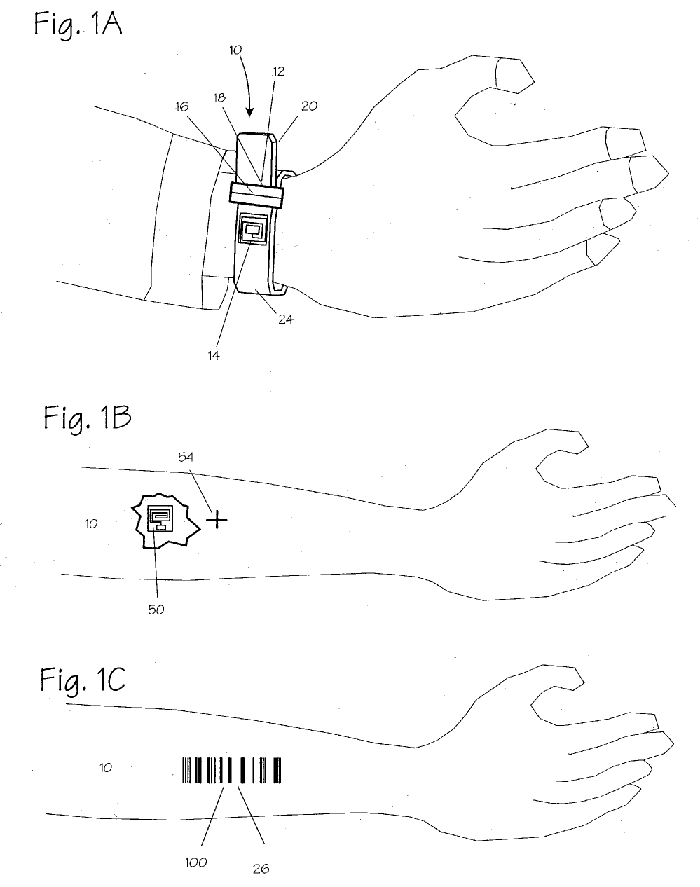

FIG. 1A illustrates a diagram of a coded device 10 of the present invention. The coded device 10 comprises a bracelet 12 and an identifying system 14. The bracelet 12 further comprises a locking mechanism 16, an adjusting mechanism 18, and a bracelet band 24 further comprising ends 20 and 22 (not shown). The identifying system 14 further comprises a code 26 (not shown).

Referring to FIG. 1A, the bracelet 12 is a linear, bendable band 24 with ends 20 and 22. The adjusting mechanism 18 is integral to or affixed to the band 24, as is the locking mechanism 16. The identifying system 14 is affixed to the band 24.

Referring to FIG. 1A, the locking mechanism 16 prevents the device to be removed from the patient, except by deliberate intent. In the preferred embodiment, the locking mechanism 16 is permanent, once locked and the bracelet band 24 must be cut to remove the bracelet 12 from the patient. The bracelet 12 is adjustable by way of an adjusting mechanism 18 that permits the two ends 20 and 22 of the bracelet band 24 to slide longitudinally with respect to each other. Once the correct size has been determined, the one-way locking mechanism 16 is engaged and prevents the bracelet 12 from being removed from the patient's wrist. The bracelet 12 is fabricated from polymers such as polypropylene, polyethylene, polyvinyl chloride and the like. The bracelet 12 is preferably sized to be placed on the wrist of a patient. Preferably, the bracelet 12 would be of variable size so as to fit a wide range of patient wrist circumferences.

The identifying system 14 is affixed to the band 24 using adhesives, injection molding, vapor deposition, inkjet printing, lithography or other technology well known in the fabrication art. Preferably, the identifying system 14 comprises the pre-determined unique code 26 that is permanently related to the band 24 of the bracelet 12. By permanently relating the bracelet 12 to the patient, the patient now retains the unique, pre-assigned identifying number.

The identifying system 14 utilizes a passive method of identification so that power is not required in order to allow for reading of the code 26. Typical identifying systems 14 for carrying the code 26 include RF ID transponders of the type used in cars to provide toll road billing information. Such RF ID transponders typically comprise a transponder chip that holds the code 26 and a metal coil that acts as an antenna for the RF input and output and power generation. Other identifying systems 14 include numerical printed codes. Optical character recognition (OCR) readers could easily read these codes. Other code systems include bar coding used on much of the packaging of products today. Bar code readers are readily seen in places like supermarkets and other store check-out lines. These marks would be placed on the bracelet 12 using indelible ink and lithography, inkjet printing, laserjet printing and the like. A transparent or semi-transparent coating or protective layer is optionally placed over the printed code 26 to further prevent it from becoming damaged or suffer from reduced legibility. Such a coating is fabricated from polyurethane, lacquer, or other transparent polymer. The coating may also be fabricated from silica substances such as glass.

The bracelet 12 represents one form of jewelry that comprises the coded device. In another embodiment, the jewelry is a necklace that is worn around the neck and comprises all the same components as the bracelet except that it is fitted to the neck of the patient. In another embodiment, the bracelet 12 is sized to fit the leg of the patient rather than the wrist. In another embodiment, the jewelry is an earring that is affixed through a hole surgically created in the ear of a patient.

FIG. 1B illustrates another embodiment of the coded device 10 using a transponder 50 implanted in the patient. The transponder 50 comprises the code 26 (not shown). The transponder 50 is a passive transponder shaped so as to be implantable. Preferred shapes include cylinders and thin, flat rectangular solids. The cylindrical transponder 50 may be loaded into an injector (similar to a hypodermic needle and syringe) that is used to create a fenestration through the patient's skin into the subcutaneous area. The transponder is then advanced through the fenestration in the skin and the injector is removed so that the transponder 50 is able to heal in place under the skin. The transponder 50 and the injector are sterile and are supplied in a sterile package so that infection is avoided. In order to further clarify the position of the transponder 50, a visible mark 54 is left on the patient's skin in the region of the transponder 50 so said transponder 50 can easily be located. Such a visible mark is created using the technology used for tattooing.

Though not preferred, the mark 100 may optionally placed into the skin using tattoo technology by injection into the skin or jewelry. Such tattooing technology provides permanent skin marking, and may be beneficial and necessary if the transponder embodiments are unworkable for any reason. FIG. 1C illustrates this embodiment of the coded device 10 wherein the coded device 10 is a mark 100 that is optically or magnetically readable and is affixed directly to the skin of the patient. The mark 100 is a plurality of alphanumeric, bar or geometric figures that comprise the code 26. In this figure, the code 26 can be visualized because it is a bar code. The mark 100 is of a color that contrasts with the skin or a surrounding background color placed adjacent to said mark 100. Indelible inks such as those with carbon solvent bases such as alcohol are used for indelible inks. Typical colors for the mark 100 include black, green, blue or colors that only reflect under specialized lighting such as ultraviolet or infrared, so that the mark is not disfiguring or embarrassing or otherwise socially undesirable to the patient.

FIG. 2 illustrates a schematic of the data encoding system 150. The data encoding system comprises a coded device writer 152, a coded device reader 154, a global positioning system 156, a clock 158, a computer 160, digital memory storage 162, a data acquisition system 164, a communication transmitter 166, a communication receiver 168, an optional set of data sensors 170, a voice input system 172, a power supply 174, a case 176, an alphanumeric keyboard 178, a video display 180, a plurality of input connectors 182, a plurality of controls 184 and an optional protective panel 186. The voice input system 172 further comprises a microphone 188. The data acquisition system 164 further comprises optional sensors 170 for detecting and reading parameters such as patient temperature, electrocardiogram, blood pressure and the like. Such sensors 170 are well known in the art. The data encoding system further comprises a unique identifier 192 that distinguishes one data encoding system from another.

Referring to FIG. 2, the data encoding system 150 is self-contained within its case 176. The coded device writer 152, the coded device reader 154, the global positioning unit 156, the clock 158, the computer 160, the digital memory storage 162, the data acquisition system 164, the communication transmitter 166, the communication receiver 168, the data sensors 170, the power supply 174, the keyboard 178 and the video display 180 are all affixed to the case 176. The video display 180, a keyboard 178, a plurality of input connectors 182, and the plurality of controls 184 are affixed to the case so that they are accessible by the user. The case 150 comprises an optional protective panel 182 that hinges and covers the video display 180, the keyboard 178, the input connectors 182 and the controls 184. Preferably, either the video display 180 or the keyboard 178 is located on the interior surface of the protective panel 182 so as to minimize the size of the device case 176. The microphone 188 and the sensors 170 are external to the case and are electrically connected to the case through the input connectors 182. The unique identifier 192 is maintained in the computer 160. The unique identifier 192 is maintained in a permanent memory such as a read only memory (ROM) or electrically programmable read only memory (EPROM) that is part of the computer 160.

The voice input system 172 allows for entry of critical information on the patient. (This information may also be entered using keyboards or touch screens or other input mechanisms.) This critical information includes, but is not limited to, a description of known or suspected injuries, contamination status, triage status, situation surrounding the incident, proposed treatment plan and proposed patient disposition. The computer 160 stores, in digital memory storage 162, the information entered through the voice input system 172 in the form of a standard digital recording in formats such as .wav or .mpg. The voice input system 172 and computer 160 optionally comprise a voice recognition system that digitizes the analog voice recording into alphanumeric text and stores said alphanumeric information in the digital memory storage 162. Various triage parameters such as heart rate, cardiac filling, breathing rate, end-tidal volume may be detected (at least roughly) using ultrasound, and other triage parameters such as EKG, EEG, and gastric rhythm may be measured with appropriate electrodes and analyzers, and these parameters may be entered into the device.

Referring to FIG. 2, the data encoding system 150 sends a request to the centralized database 200 for the next assignable code 26. The request includes the unique identifier 192 for the data encoding system 150 and is sent to the centralized database by the communication transmitter 166. The data encoding system 150 receives the next assignable code 26 through the communication receiver 168 from the centralized database 200. The coded device writer 152 applies the code to the coded device 10. The coded device reader 154 next reads the coded device 10 and records the code 26 in digital memory storage 162. Patient information is gathered through the keyboard 178 by the attending medical personnel. Additional patient information is obtained from the clock 158 and the Global Positioning System 156 as well as a microphone 188 electrically connected to the voice input system 172 through input connectors 182 and information from all three sources is stored by the computer 160 in the digital memory storage 162. Additional information is received through the data acquisition system 164 by its plurality of sensors 170 and this information is stored in the digital memory storage 162 by the computer 160.

Referring to FIG. 2, the information stored in digital memory storage 162 is transmitted to the central database by the communication transmitter 166. The communication transmitter 166 operates over typical protocols including, but not limited to, CDMA, TDMA, PCI, cell phone or satellite communication protocols. The communication receiver 168 receives information already stored in the central database, relating to the code 26 that was obtained by the coded device reader 154.

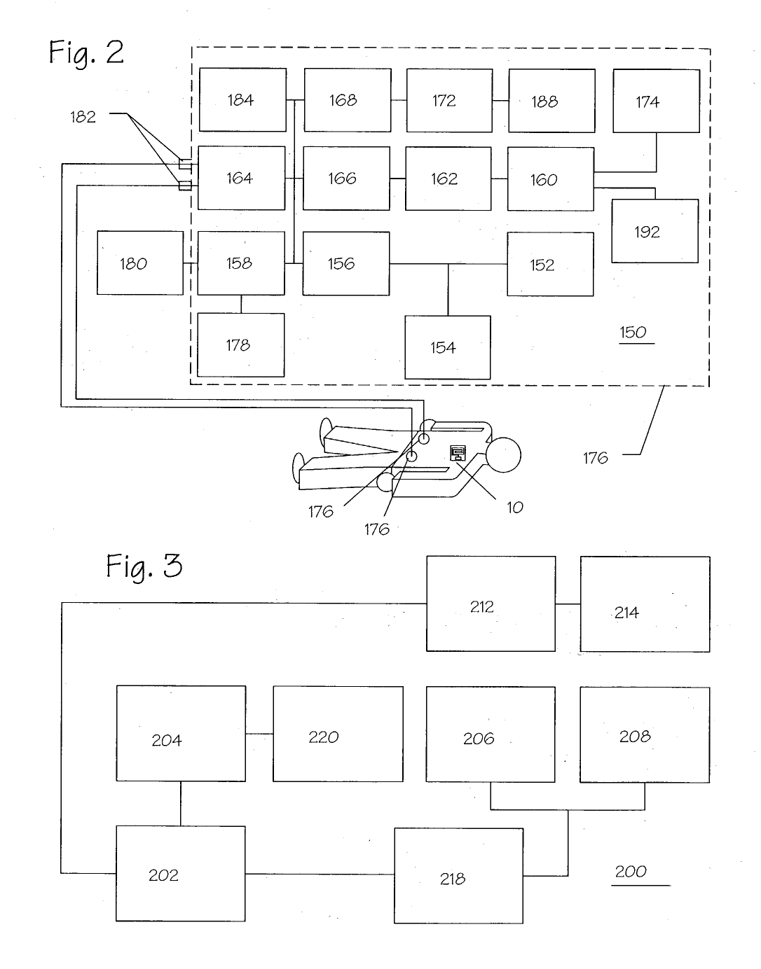

FIG. 3 illustrates a schematic diagram of the centralized database 200. The centralized database 200 comprises a plurality of computers 202, a plurality of storage devices 204, a plurality of backup devices 220, a communication transmitter 206, a communication receiver 208, a communication interface 218, software 210 (not shown) to run the database 200, a power source 212, a plurality of uninterruptible power supplies 214, and data 216 (not shown).

Referring to FIGS. 2 and 3, the centralized database 200 maintains a list of the already assigned codes 26 (not shown) and maintains the protocol for assigning new codes 26. When a request comes in over the communication receiver 208, and is passed on through the communications interface 218, the next assignable code 26 is generated by the computers 202 based on the data 216 (not shown) already maintained in the storage devices 204. The assignable code 26 is related to the unique identifier 192 of the data encoding system 150, then stored in the storage devices 204, and then processed through the communications interface 218 and transmitted over the communications transmitter 206 to the communication receiver 168 of the data encoding system 150. Future data relayed to the centralized database 200 will include the unique identifier 192 and the code 26 so that the patient can be tracked and information collection related to the team performing the service through their unique identifier 192. All such future data relayed to the central database is maintained in association with the code 26.

The overall operation of the system will include cooperation amongst many health care providers, all of whom can access the central database and query the transponder or identifying device or mark, and obtain triage information and patient history from the database or transponder. Additionally, at each encounter with a health care provider, patient condition can be assessed and entered into the database, so that population-wide development of a disease, exposure or catastrophic event, to use in epidemiological analysis of the population. Thus, changing symptoms of the patients at each encounter can be tracked and used to determine disease vectors, contamination sources, and the expected spread and future development of the condition. Thus, a vector or origin of contaminants or injury to a patient can be determined upon later encounters with health care providers.

The present invention may be embodied in other specific forms without departing from its spirit or essential characteristics. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

What is claimed is:

1. An apparatus adapted for patient tracking comprising:
a unique code associated with the patient;
a device that passively retains said unique code;
a device that affixes said unique code to a patient;,
a device that reads the unique code;
a database that retains patient information related to the unique code; and
a system that links said device to read the code to said database.

2. The apparatus of claim 1 wherein said device that passively retains said unique code is a tattoo or other mark on the skin of the patient.

3. The apparatus of claim 1 wherein said device that passively retains said unique code is a passive transponder.

4. The apparatus of claim 1 wherein said device that passively retains said unique code is a piece of jewelry further comprising a transponder or optically readable mark.

5. A method of patient tracking that involves the steps of:
Assigning a unique code to a patient,
Affixing said code to said patient;
Determining physiological parameters relevant to a medical condition of the patient;
Entering the patient's physiological data into a database at the time the unique code is affixed to said patient,
Reading said code at a future time to determine the identity of said patient and,
Utilizing, augmenting and updating said data to assist with treatment of said patient.

6. The method of claim 5 wherein said data includes geographic position as determined by a GPS system.

7. The method of claim 5 wherein said data includes time information.

8. The method of claim 5 wherein said data includes the unique identifier of the team relaying the information to the database.

9. The method of claim 5 wherein said data includes physiological parameters measured on the patient.

10. The method of claim 5 further comprising the step of determining physiological parameters is accomplished by ultrasound.

11. The method of claim 5 further comprising the step of assigning a triage score to the patient and entering the score in the database.

12. The method of claim 5 further comprising the step of determining a suspected event causing relevant symptoms in the patient and entering this information in the database.

13. The method of claim 13 further comprising the step of analyzing the data entered for a plurality of patients to determine a common cause of symptoms occurring in the plurality of patients.
